(19) United States
(12) Patent Application Publication
(10) Pub. No.: US 2012/0199137 A1
(43) Pub. Date: Aug. 9, 2012

(54) VACUUM ASSISTED MOUTHPIECE

(76) Inventor: Nicholas G. Zacharopoulos, New City, NY (US)

(21) Appl. No.: 13/022,169

(22) Filed: Feb. 7, 2011

(51) Int. Cl. A61F 11/00

(52) U.S. Cl. 128/857

(57) ABSTRACT

A mouthpiece used for head fixation use in medical procedures. The mouthpiece incorporates a vacuum cavity above the vacuum port of the mouthpiece and a vacuum channel embedded in the mouthpiece to allow connection to a vacuum hose external to patient's mouth.

### BACKGROUND

Head fixation is a noninvasive procedure used in order to prevent or minimize involuntary patient movements during various medical procedures. The patient's head is immobilized by preventing movement of a mouthpiece attached to the palate of the patient's mouth and teeth. The patient's teeth are the only bony protrusions rigidly connected to the cranium. The palate is covered with relatively non-elastic soft tissues that retain shape. Together they provide rigid interface for the mouthpiece.

During the preparation a process patient-specific mouthpiece is created using dental impression material. This mouthpiece conforms to the anatomy of the patient's palate and teeth. The mouthpiece also incorporates a vacuum cavity above the vacuum port of the mouthpiece. By applying vacuum, the mouthpiece becomes securely attached to the patient's cranium.

As shown on FIGS. 1-3, a traditional vacuum-assisted mouthpiece (1) has two interface prongs (2), a vacuum port (3) on top of the mouthpiece, corresponding vacuum port (4) on bottom of the mouthpiece, and a vacuum tube (5) connected to the bottom side port (4) for a vacuum. The mouthpiece is manufactured as a single piece.

The major disadvantage of this typical mouthpiece is that the vacuum tube is connected to the port inside of the patient's mouth. (The vacuum tube is slipped onto the tapered tube port prior to the insertion of the mouthpiece into the patient's mouth.) The vacuum tube is guided out of the patient's mouth and into the fluid collection unit. The following issues may arise during use:
1. Inadvertent disengaging of the vacuum hose. Since the hose may be in contact with the tongue, the patient can loosen and disengage the hose from the vacuum port. This would eliminate the vacuum and the fixation.
2. Any intervention regarding the vacuum tube connection requires that the patient is released from the device and the mouthpiece is removed from the patient's mouth.
3. Reduced ability to communicate. Since only upper jaw is immobilized, the patient retains ability to communicate and swallow any saliva. However, the vacuum tube protruding through the mouth further reduces patient's ability for verbal communication
4. Obstructions to other medical devices. Some patient's are intubated during the procedures. An additional tube further complicates the procedure.
5. Reduces applications of some procedures. Some treatment types require tongue depressors or immobilization of the lower jaw. The tube going through the patient's mouth reduces the range of possible techniques.
6. Patient comfort. Tube inside of the patient's mouth reduces patient comfort during the procedures, increase the potential for gagging or severity of gagging.

### SUMMARY

A mouthpiece in which the evacuation channel is incorporated into the mouthpiece itself.

### BRIEF DESCRIPTION OF DRAWINGS

FIG. 1 depicts a top perspective view of a typical existing mouthpiece.

FIG. 2 depicts a bottom perspective view of a typical existing mouthpiece.

FIG. 3 depicts a bottom perspective view of a typical existing mouthpiece with a vacuum hose attached.

FIG. 4 depicts a top perspective view of an improved mouthpiece with an embedded vacuum channel.

FIG. 5 depicts a bottom perspective view of mouthpiece with an embedded vacuum channel.

FIG. 6 depicts an improved mouthpiece with its two sections separated from each other.

FIG. 7 depicts an improved mouthpiece with the vacuum hose connected.

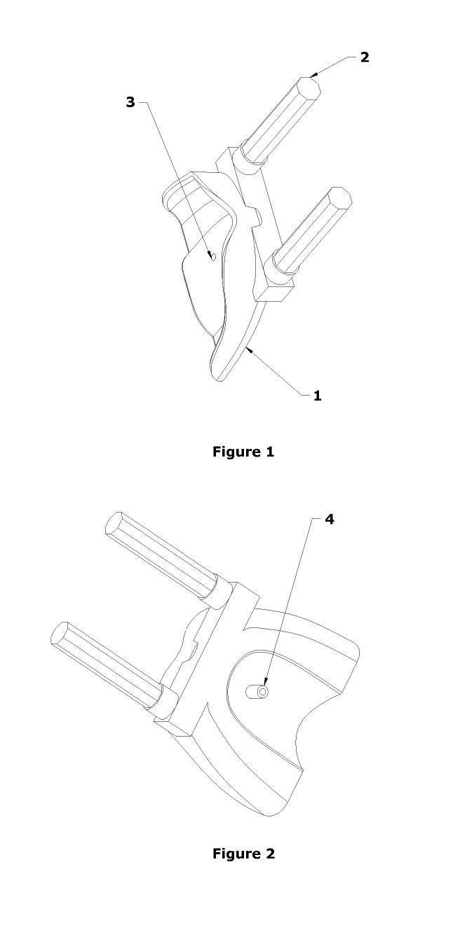

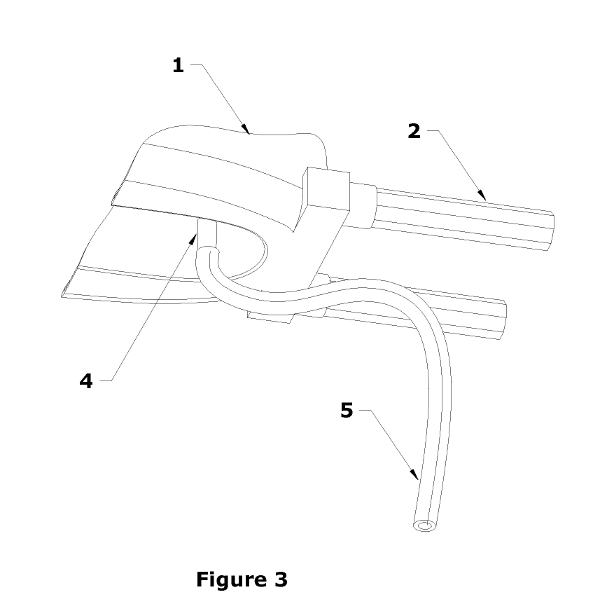

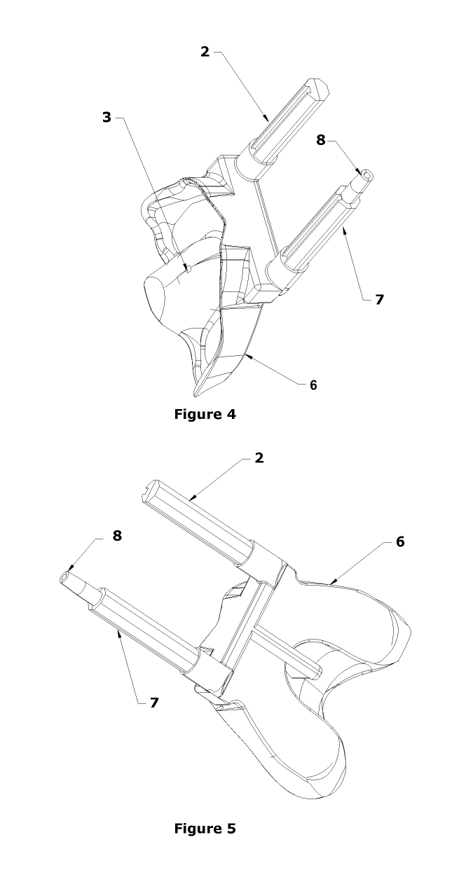

### DETAILED DESCRIPTION

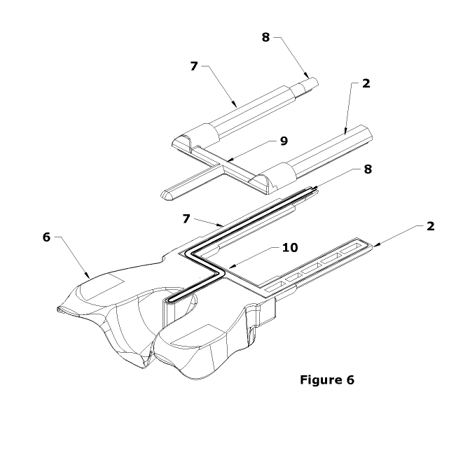

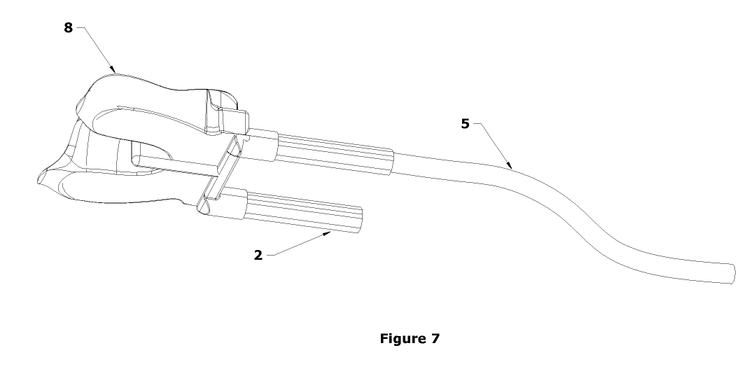

As depicted on FIGS. 4-7, the improved mouthpiece (6) includes a standard interface prong (2), an interface prong (7) with embedded vacuum channel (8), a vacuum port (3) and a vacuum hose (5). The mouthpiece is constructed by adhesively jointing together an upper port (9) to a lower port (10) as depicted on FIG. 6 (for example, the ports may be welded together). This allows the creation of a sealed pathway starting at the interface of the mouthpiece with the palate at vacuum port (3) (where vacuum is created) and ending at the tube interface with the vacuum hose at the end of prong (7) with the embedded vacuum channel (8). A vacuum cavity can thus be formed between the mouthpiece and a patient's palate.

The above illustrates various concepts, structures and techniques which are the subject of this patent. It will now become apparent to those of ordinary skill in the art that other embodiments incorporating these concepts, structures and techniques may be used. Accordingly, it is submitted that that scope of the patent should not be limited to the described embodiments but rather should be limited only by the spirit and scope of the following claims. For example, evacuation channels may be incorporated into both prongs and connected to separate vacuum hoses or even separate vacuum sources.

1. A mouthpiece for vacuum assisted head fixation comprising:
a mouthpiece body;
two interface prongs wherein at least one prong includes a vacuum channel and designed for connection to a vacuum hose;
a vacuum port designed to create a vacuum cavity between the mouthpiece and a patient's palate when vacuum is applied to the vacuum channel.

2. A mouthpiece according to claim 1 wherein the mouthpiece body is constructed of an upper port and a lower port and adhesively joined together.

3. A mouthpiece according to claim 2 wherein the two ports are adhesively joined together by welding.

4. A method utilizing a vacuum assisted head fixation mouthpiece comprising:
selecting a mouthpiece wherein a vacuum channel is embedded into the mouthpiece with a vacuum connection port designed to be located external to a patient's mouth;
connecting a vacuum hose to the connection port;
inserting the mouthpiece into a patient's mouth;
positioning the mouthpiece; and
turning on a vacuum source to the hose.
